(19) United States
(12) Plant Patent Application Publication
(10) Pub. No.: US 2006/0225172 P1
(43) Pub. Date: Oct. 5, 2006

(54) CHRYSANTHEMUM PLANT NAMED 'MOZA THREE ANGA'

(50) CHRYSANTHEMUM MORIFOLIUM; CHRYSANTHEMUM

(76) Inventor: Frank Moser, Alva, FL (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/095,432

(22) Filed: Mar. 30, 2005

(51) Int. Cl. A01H 5/00

(52) U.S. Cl. PLT/294

(57) ABSTRACT

A new and distinct Chrysanthemum plant cultivar is disclosed, characterized by moderate to rapid vigor and the presence of white, long lasting, daisy-like flowers that have a very small disc. The plant has a mounding, spreading growth habit, and is free-branching.

### LATIN NAME OF THE GENUS AND SPECIES

Chrysanthemum morifolium

### VARIETY DENOMINATION

‘MOZA THREE ANGA’

### BACKGROUND OF THE INVENTION

The new cultivar is a product of hybridization of a female parent Chrysanthemum plant variety ‘MZ 133’ (undistibuted seedling) and a male parent Chrysanthemum plant variety ‘MZ 08’ (undistributed seedling). The new cultivar was discovered and selected by Frank Moser in September, 2000 in Alva, Fla.

Asexual reproduction of the new cultivar ‘MOZA THREE ANGA’ by terminal cuttings and tissue culture was performed in Oxnard, Calif., and has shown that the unique features of this cultivar are stable and reproduced true to type on successive generations.

### SUMMARY OF THE INVENTION

The cultivar ‘MOZA THREE ANGA’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘MOZA THREE ANGA.’ These characteristics in combination distinguish ‘MOZA THREE ANGA’ as a new and distinct Chrysanthemum cultivar:

1. The flower is a white daisy with a very small disc.

2. The plant has a mounding, spreading growth habit.

3. The plant has a free-branching habit.

4. The floral longevity is approximately 28 days.

5. The plant vigor is moderate to rapid.

6. The plant naturally blooms beginning the last week of August or first week of September.

Plants of the new cultivar ‘MOZA THREE ANGA’ are similar to plants of the female parent variety, ‘MZ 133’ (undistibuted seedling) in most horticultural characteristics, however the new cultivar has white florets, while the female parent variety has pink florets. The flower of the new variety ‘MOZA THREE ANGA’ has a higher longevity than the flower of the female parent ‘MZ 133’ by approximately seven days. Additionally, the plant of the new variety ‘MOZA THREE ANGA’ has a shorter growth habit than the female parent ‘MZ 133.’

Plants of the new cultivar ‘MOZA THREE ANGA’ are similar to plants of the male parent variety, ‘MZ 08’ (undistributed seedling), in most horticultural characteristics, however plants of the new cultivar ‘MOZA THREE ANGA’ have a daisy-type flower, while the plants of the male parent variety have a decorative-type flower. The new variety ‘MOZA THREE ANGA’ naturally blooms approximately three weeks earlier than the male parent variety ‘MZ 08.’ Further, the new variety ‘MOZA THREE ANGA’ is taller than the male parent variety ‘MZ 08.’

In comparison to the commercially available variety ‘GEDI WT8’ (U.S. Plant Pat. No. 11,648), ‘MOZA THREE ANGA’ has a smaller disc center and more ray florets. The floral longevity of the new variety is approximately nine days longer than the floral longevity of the comparable variety ‘GEDI WT8.’ The new variety has about 17 more flowers per stem than the comparable variety ‘GEDI WT8.’ Additionally, the new variety ‘MOZA THREE ANGA’ blooms earlier than does the comparable variety.

In comparison to the commercially available variety ‘PIDOUL’ (U.S. Plant Pat. No. 10,218), ‘MOZA THREE ANGA’ has more ray florets per flower. The new variety also has a larger diameter flower that blooms about seven days earlier than the comparable variety ‘PIDOUL.’ Additionally, the new variety ‘MOZA THREE ANGA’ has a more flexible plant habit which is more resistant to breakage than the comparable variety ‘PIDOUL.’

### BRIEF DESCRIPTION OF THE PHOTOGRAPH

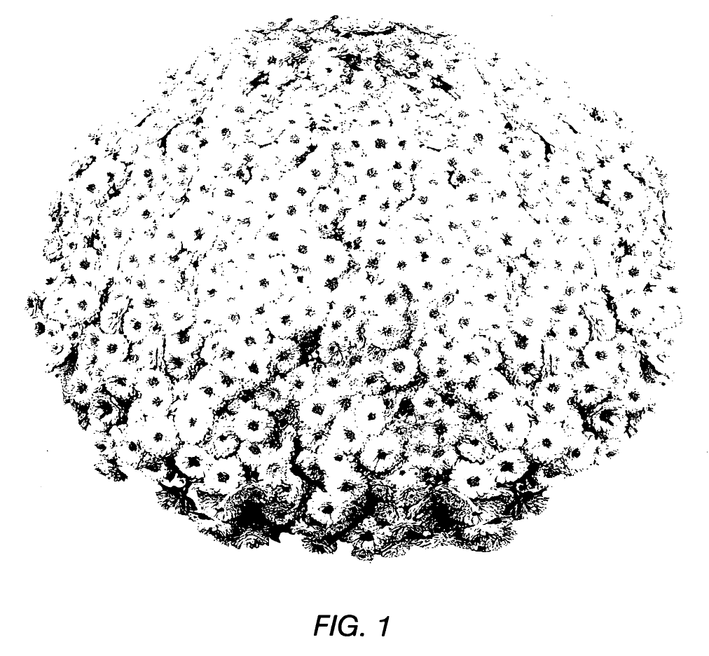

The accompanying photograph in FIG. 1 illustrates in full color a typical plant of ‘MOZA THREE ANGA’ grown in a 2 gallon container. One cutting was used in the pot. The colors are as nearly true as is reasonably possible in a color representation of this type.

### DETAILED BOTANICAL DESCRIPTION

In the following description, color references are made to The Royal Horticultural Society Colour Chart except where general terms of ordinary dictionary significance are used. The following observations and measurements describe ‘MOZA THREE ANGA’ plants grown in a greenhouse in Oxnard, Calif. during the month of April, 2004. The growing temperature ranged from about 12° C. to 15° C. at night to about 12° C. to 20° C. during the day. Measurements and numerical values represent averages of typical flowering types, and are believed to apply to plants of the variety grown under similar conditions of soil and climate elsewhere:
Botanical classification: Chrysanthemum morifolium ‘MOZA THREE ANGA.’
Commercial classification: Garden-type Chrysanthemum.

### Propagation

Time to rooting: 7 to 14 days at approximately 21° C.
Root description: Fine, fibrous.

### Plant

Growth habit: Mounding herbaceous perennial.
Height: Approximately 30 cm.
Plant spread: Approximately 34 cm.
Growth rate: Moderate.
Branching characteristics: Free branching.
Length of lateral branches: Approximately 28 cm.
Number of leaves per lateral branch: Approximately 23.
Age of plant described: Approximately 135 days from a rooted cutting.

### Foliage

Leaf:
   Arrangement.—Alternate.
   Average length.—Approximately 6.5 cm.
   Average width.—Approximately 5.6 cm.
   Shape of blade.—Ovate.
   Apex.—Cuspidate.
   Base.—Attenuate.
   Attachment.—Stalked.
   Margin.—Palmately lobed and toothed.
   Texture of top surface.—Lightly pubescent.
   Texture of bottom surface.—Lightly pubescent.
   Leaf internode length.—Approximately 2.7 cm.
   Color.—Young foliage upper side: Near Green 138A. Young foliage under side: Near Green 138A. Mature foliage upper side: Near Green 137C. Mature foliage under side: Near Green 137D.
   Venation.—Type: Palmately net. Venation color upper side: Near Green 138B. Venation color under side: Near Green 138B.
Petiole:
   Average length.—Approximately 1.0 cm.
   Color.—Near Green 138B.
   Diameter.—Approximately 0.2 cm.

### Bloom

Flower:
   Flowering habit.—Induced by darkness period greater than approximately 13.5 hours, approximately 49 days of appropriate day length required to induce and develop blooms.
   Form.—Daisy.
   Natural flowering season.—Approximately the last week of August and first week of September.
   Number of flowers per lateral branch.—Approximately 29.
   Diameter.—Approximately 4.7 cm.
   Disc diameter.—Approximately 1 cm.
   Depth.—Approximately 2.1 cm.
   Longevity on plant.—Approximately 28 days.
   Persistence.—Persistent.
Ray florets:
   Appearance.—Matte.
   Texture.—Smooth.
   Average number of ray florets per flower.—Approximately 195.
   Shape.—Oblanceolate.
   Aspect.—Flat.
   Margin.—Entire.
   Apex.—Retuse.
   Length.—Approximately 1.5 cm.
   Width.—Approximately 0.8 cm.
   Color.—Upper surface at first opening: Near Yellow 8D. Upper surface at maturity: Near White 155D. Upper surface at fading: Near White 155D with some ray florets showing near Red-purple 63C at apex. Under surface at first opening: Near White 155D. Under surface at maturity: Near White 155D. Under surface at fading: Near White 155D with some ray florets showing near Red-purple 63C at apex.
Disc Florets:
   Appearance.—Shiny.
   Texture.—Smooth.
   Average number of florets per disc.—Approximately 50.
   Shape.—Cylindric.
   Apex.—Obtuse.
   Average length.—Approximately 0.8 cm.
   Average width.—Approximately 0.1 cm.
   Color.—At first opening: Near Yellow-orange 14A. At maturity: Near Yellow-orange 17A. At fading: Near Yellow-orange 22A.
Peduncle:
   Length.—At terminal end (shortest): Approximately 4.5 cm. At lateral end (longest): Approximately 7.1 cm.
   Angle to stem.—Acute.
   Strength.—Moderate.
   Color.—Near Green 138A.
   Habit.—Upright.
   Diameter.—Approximately 0.2 cm.
   Surface texture.—Lightly pubescent.
Flower bud:
   Length.—Approximately 0.8 cm.
   Diameter.—Approximately 0.8 cm.
   Form.—Globular.
   Color.—Near Yellow 8D.
Involucral bracts (phyllaries):
   Appearance.—Matte.
   Texture.—Lightly pubescent.
   Number.—Approximately 35.
   Shape.—Oblanceolate.
   Margin.—Entire.
   Apex.—Acute.
   Length.—Approximately 0.8 cm.
   Width.—Approximately 0.8 cm.
   Color.—Upper side: Near Green 138A. Under side: Near Green 138A.

### Reproductive Organs

Ray florets:
   Number of pistils per floret.—1.
   Stigma shape.—2 branched.
   Stigma color.—Near Yellow 2C.
   Style color.—Near Yellow-green 154D.
   Style length.—Approximately 0.7 cm.
   Stamens.—Absent.
Disc florets:
   Number of pistils per floret.—1.
   Stigma shape.—Two-branched.
   Stigma color.—Near Yellow 2A.
   Style length.—Approximately 0.2 cm.
   Style color.—Near Yellow 2D.
   Number of stamens per floret.—Approximately 5.
   Anther shape.—Tubular.
   Anther color.—Near Yellow 12A.
   Pollen color.—Near Yellow-orange 15A.

### OTHER CHARACTERISTICS

Disease resistance: Neither resistance nor susceptibility to diseases or pests has been observed in this variety.
Drought tolerance/cold tolerance: Flowering plants are hardy to low temperature about −2° C. Non-flowering plants are hardy in the approximate range of 3° C. to −6° C., depending upon duration of cold an amount of moisture in the soil. With adequate water plants are hardy to a high temperature of 45° C.
Fruit/seed production: Commercially, this plant is not used or observed in a stage wherein seeds would be produced. Therefore, seed production has not been observed.

What is claimed is:

1. A new and distinct cultivar of Chrysanthemum plant named ‘MOZA THREE ANGA’ as herein illustrated and described.
